(19) United States
(12) Patent Application Publication
(10) Pub. No.: US 2014/0309598 A1
(43) Pub. Date: Oct. 16, 2014

(54) OSMOTICALLY ACTIVE VAGINAL DELIVERY SYSTEM

(71) Applicant: BAYER OY, Turku (FI)

(72) Inventors: Manja Ahola, Piikkio (FI); Stefan Bracht, Glienicke/Nordbahn (DE); Harri Jukarainen, Kuusisto (FI); Pirjo Kortesuo, Parainen (FI); Heikki Lyytikäinen, Naantali (FI); Mikael Stolt, Vahto (FI)

(73) Assignee: BAYER OY, Turku (FI)

(21) Appl. No.: 14/356,159

(22) PCT Filed: Nov. 1, 2012
(86) PCT No.: PCT/FI2012/051064

§ 371 Date: May 4, 2014

(30) Foreign Application Priority Data
Nov. 1, 2011 (FI) 20116073

(51) Int. Cl. A61K 9/00

(52) U.S. Cl. CPC A61K 9/0004; A61K 9/0036
USPC 604/285

(57) ABSTRACT

The present invention relates to the field of drug delivery systems. More particularly, the invention relates to osmotically active intravaginal delivery systems for the controlled release of therapeutically active substances to the vaginal cavity.

The present invention relates to the field of drug delivery systems. More particularly, the invention relates to osmotically active intravaginal delivery systems for the controlled release of therapeutically active substances to the vaginal cavity.

### BACKGROUND OF THE INVENTION

Vaginal rings are an attractive form of medical device for local or systemic release of one or more pharmaceutical active substances in the female vaginal region. The systems are suitable for self-application and also self-removal by the female. Diffusion-controlled systems are successful and have been widely described in the literature.

In vaginal rings that contain the active substance in dissolved form, the release of the active substance takes place principally according to Fick's first law of diffusion. In a system containing a suspended, undissolved active substance, the transport of the substance over time is governed by the Higuchi equation:

$$\frac{d {M}_{t}}{d t} = \frac{A}{2} \sqrt{\frac{2 {DC}_{S} {C}_{0}}{t}}$$

wherein Mₜ is the amount of active substance which will be released in time t, D is diffusion coefficient of the active substance through the polymer, C₀ is the total concentration of the drug in the carrier matrix, Cₛ is the solubility of the drug in the polymer and A is the area through which the substance diffuses.

When applied to dissolution Fick's law may be expressed as follows

$$\frac{d {M}_{t}}{d t} = \frac{DA ( {C}_{S} - {C}_{b} )}{h}$$

where D is the diffusion coefficient, A the surface area, Cₛ the solubility of the drug in the polymer, C_{b} the concentration of drug in the bulk and h the thickness of the diffusion layer. If C_{b} is much smaller than Cₛ then we have so-called “sink conditions” and the equation reduces to

$$\frac{d {M}_{t}}{d t} = \frac{{DAC}_{S}}{h}$$

Fick's law suggests that the rate of diffusion in a given direction across the surface is directly proportional to the concentration gradient—the steeper the concentration gradient, the faster the rate of diffusion. The rate of diffusion is directly proportional to the surface area—the greater the surface area of a membrane through which diffusion is taking place, the faster the rate of diffusion. This is one of the factors which limit cell size. Finally, the rate of diffusion is inversely proportional to the distance—the rate of diffusion decreases rapidly with distance. Diffusion is thus effective only over short distances.

In both cases, high rates of active substance release per unit of time require at least one of the following conditions:
large system surface
high coefficient of diffusion of the active substance
high concentration gradient between system surface and application site

Due to different diffusivity, the release rate of certain pharmaceutically active substances through polymers per unit of time may be limited in diffusion-controlled systems. For example, relatively water-soluble drugs or drugs having too large molecular size/volume/weight may not be soluble enough in the polymer material to permit sufficient drug release.

Several strategies have been described to achieve the release of relatively hydrophilic substances, water-soluble drugs or macromolecular agents at therapeutic concentrations.

The polymer material can be modified to increase the solubility of hydrophilic substances in hydrophobic polymers.

In a matrix system the drug substance can be loaded at very high concentrations (over 20% w/w). In such a system, the drug substance is distributed throughout the device. The combination of high loading and the availability of the drug substance on the surface of the ring device results in relatively high release rates, at least during the initial period after application. However, it is not cost effective to incorporate potent and expensive therapeutic macromolecules or water-soluble drugs into matrix rings at such high loadings. Since release takes place from the surface of the device, a significant proportion of the drug substance within the bulk of the matrix ring may never be released, but will be retained within the bulk of the ring.

Water-soluble release enhancers can be incorporated into matrix rings such that water/fluid uptake into the ring promotes the release of the incorporated water-soluble or macromolecular agents. However, high loadings of the water-soluble release enhancers are required to significantly enhance the release rate of the drug substance. Additionally, the subsequent water/fluid uptake by the water-soluble release enhancer within the device may lead to excessive swelling and expansion of the device such that its original shape and size are no longer maintained. Such swelling and expansion would place excessive pressure on the vaginal walls, making the device unsuitable for use.

Sustained release of water-soluble or macromolecular agents has been obtained from subcutaneously implantable devices, wherein the water-soluble drug or macromolecule and a water-soluble release enhancer are incorporated into a silicone elastomer core which is partially encapsulated with a polymeric sheath, such that the ends of the core containing the drug substance and the release enhancer are exposed to the external environment. (M. Kajihara et al, J. Cont. ReI. 66 (2000) 49-61; M. Kajihara et al, J. Cont. ReI. 73 (2001) 279-291; J. M. Kemp et al., Vaccine 20 (2002) 1089-1098; S. A. Lofthouse et al., Vaccine 20 (2002) 1725-1732; M. Maeda et al., J. Cont. ReI. 84 (2002) 15-25; H. Maeda et al., Int'l. J. Pharm. 261 (2003) 9-19; M. Kajihara et al., Chem. Pharm. Bull. 51 (2003) 15-19; H. Maeda et al., J. Cont. ReI. 90 (2003) 59-70.) The release of the drug substance is achieved through uptake of the surrounding medium or bodily fluid into the core, followed by dissolution and removal of the water-soluble release enhancer, and concomitant dissolution and release of the drug substance. From the perspective of vaginal administration of drug substances, the device, which has been specifically developed to be implanted into the tissue, is not likely to be retained within the vagina owing to its size and shape of construction.

International patent application WO 2009003125 by Warner-Chilcott relates to an intravaginal drug delivery device comprising a hydrophobic carrier material having at least one channel defining at least one opening to the exterior of said device body. The at least one channel is adapted to receive at least one drug-containing insert which is capable of releasing a pharmaceutically effective amount of at least one drug suitable for intravaginal administration and containing about 1% to about 70% of at least one water-soluble release enhancer. The drug and the water-soluble release enhancer are dispersed in an insert carrier material, which may be the same or different as the hydrophobic carrier material. The at least one drug-containing insert is exposed on said exterior of said device body when said intravaginal drug delivery device is in use.

Osmotically active systems represent an alternative to diffusion-controlled active substance release systems. For example U.S. Pat. No. 4,765,989 by Alza relates to an osmotic device comprising a wall that surrounds a compartment comprising: a first osmotic composition comprising a beneficial agent, and an osmopolymer and optionally an osmagent, said composition in contacting arrangement with (2) a second composition comprising an osmopolymer and optionally an osmagent. At least one passageway through the wall connects the exterior of the osmotic device with the first osmotic composition containing the beneficial agent for delivering the beneficial agent from the osmotic device. The osmotic device is preferably useful for delivering (3) beneficial agents that because of their solubilities are difficult to deliver in a known amount at a controlled rate from an osmotic dispensing system, and for delivering (4) beneficial agents that are therapeutically very active and are dispensed in small amounts at a controlled rate from the osmotic dispensing system.

Osmotically active systems, for vaginal use among other uses, were described in principle as early as 1974 by Theeuwes and Higuchi for ALZA Corp. in U.S. Pat. No. 3,845,770, but they were not exploited commercially, as far as the inventors are aware. By contrast, osmotic systems for oral and gastrointestinal use have been developed.

### OBJECT OF THE INVENTION

An object of the invention is to provide an osmotically active vaginal delivery system, the body of which comprises
at least one compartment comprising a composition of one or more therapeutically active substances
at least one compartment, either the same or different from the one comprising the therapeutically active substance(s), which comprises an osmotical composition capable to interact with water and/or aqueous biological fluids to create a concentration gradient against the exterior fluid or to swell or expand to create osmotic pressure
at least one passageway which extends from the compartment comprising the composition of one or more therapeutically active substance(s) to the outer surface of the body, and
optionally one or more membranes each covering at least part of the delivery system wherein the membrane is made of polymer composition which is permeable to the passage of water or external aqueous fluid present in the vaginal cavity but is impermeable to the compositions inside the system.

A further object of the invention is to provide an osmotically active vaginal delivery system capable of releasing a pharmaceutically effective amount of at least one therapeutically active substance suitable for intravaginal administration over relatively long periods of time, for example, multiple days or weeks, including 1-7 days, 1-14 days or 1-28 days, or longer, thereby reducing the dosing frequency. As used herein, the term “pharmaceutically effective amount” refers to an amount of a drug required to bring about a desired prophylactic or therapeutic result.

Accordingly, it is an object of this invention to provide an osmotically active vaginal delivery system for the controlled delivery of a beneficial agent to the vaginal cavity of an animal, and in particular a human, for an extended period of time.

The user will be able temporarily and for a short time to remove the delivery system out of the vaginal region, so that no appreciable amounts of active substance will be released from the system while removed, but still the release of active substance will continue soon after the system has been reinserted into the vaginal region.

All of these objects are achieved surprisingly simply by choosing an osmotically active system of the present invention.

### DESCRIPTION OF THE FIGURES

FIG. 1 illustrates a vaginal delivery system comprising two compartments, in the state prior to vaginal use. A composition (1) comprising the therapeutically active substance is located in the compartment (2). A swellable or expandable composition (3) free of active substance is located in the compartment (4). The compartments (2, 4) are connected to each other by tubular polymer segments (5), which cover selected parts of the delivery system, for example by inserting the end-pieces of the compartments (2, 4) into the segments of a tubular membrane having essentially equal or slightly greater inner diameter than the outer diameter of the compartments and by completely sealing the ends by a composite adhesive (6). At least one outlet passageway (7) is provided more or less centrally in the compartment comprising the therapeutically active substance(s).

FIG. 2 illustrates the system of FIG. 1 in a state during vaginal use. The swellable composition (3) has imbibed water or body fluid and greatly expanded towards (into) the other compartment (2) and in doing so has forced the composition (1) with the active substance out of the system through the passageway (7).

FIG. 3 likewise illustrates a vaginal delivery system comprising two compartments in the state prior to vaginal use. A composition (1) comprising an active substance is located in the compartment (2). A swellable composition (3) free of active substance is located in the compartment (4). The two compartments (2, 4) are connected to each other by two tubulat polymer segments, which partly cover the delivery system. The compartment 2 is provided with an outlet passageway (7) which, unlike in FIG. 1, is located at one end of this compartment. A barrier layer (8) at the site near the opening (7) prevents direct contact of the two compositions (1, 3).

FIG. 4 illustrates the system of FIG. 3 in a state during vaginal use. The swellable composition (3) has imbibed water or body fluid and greatly expanded towards (into) the other compartment (2) and in doing so has forced the composition (1) with the active substance out of the system through the passageway (7). The swellable formulation (3) has penetrated only from one side into the compartment (1), because the penetration to the other direction was prevented by the barrier layer (8) at the other end.

FIG. 5 illustrates a system constructed according to the principles of the system in FIG. 1, but with the difference that the two compartments (2,4) are connected by modified intermediate pieces (9) in a manner that avoids direct contact of the compositions (1,3) at the connection points by an air gap (10).

FIG. 6 illustrates a vaginal delivery system comprising one compartment (1) and in the state prior to vaginal use. A swellable composition (2) is located at one end of the compartment, whereas most part of the tube is filled with a composition comprising a therapeutically active agent (3). The ends of the compartment (1) have been connected to each other by using a tubular polymer segment (11) covering a part of the delivery system. The other end of the compartment, the end farther from the swellable composition, comprises a passageway (7).

FIG. 7 illustrates a vaginal delivery system presented in FIG. 6 during vaginal use. The swellable composition (2) has expanded through water absorption and has resulted in the release of the active substance through the passageway (7).

FIG. 8 illustrates a vaginal delivery system comprising one compartment (1) in the state prior to vaginal use. A swellable composition (2) is located at one end of the compartment, whereas most part of the compartment is filled with a composition comprising a pharmaceutically active agent (3). Both ends of the compartment have been closed by a plug (12), and in addition connected to each other by using a tubular polymer segment (5), which partly covers the delivery system. The other end of the compartment, the end farther from the swellable composition, comprises a passageway (7). As a special feature the swellable composition and the composition containing the active substance have been separated by a movable plug, here in the form of a ball (13).

FIG. 9 illustrates a vaginal delivery system presented in FIG. 8 during vaginal use. The swellable composition (2) has expanded through water absorption and has resulted in the release of a certain amount of active substance out of the system through the passageway (7). The swellable composition, when expanding, has pushed the plug (13) towards the passageway (7).

FIG. 10 illustrates a vaginal delivery system (1), wherein the composition containing the therapeutically active substance and the osmotic composition are both located in the compartments (14a and 14b) inside the tubular polymer segments (5), which partly cover the delivery system. Outlet passageways (7) are provided leading from the compartments to the outer surface of the delivery system. The remaining body of the delivery system may at least partly comprise a polymer composition. The compartments can be filled or refilled by injecting the compositions through the membranes.

FIG. 11 illustrates a vaginal delivery system (1), wherein the composition containing the therapeutically active substance and the osmotic composition are both located in the same compartment (14). In this case the composition has been pressed to a solid form having a preselected shape and dimension that correspond to the internal dimensions of the body. Outlet passageways (7) are provided leading from the compartment to the outer surface of the delivery system. The remaining body of the delivery system may at least partly comprise a polymer composition.

FIGS. 12-15 illustrate release profiles obtained by vaginal delivery systems prepared according to Example 5. The release tests are discussed in Examples 6 to 9.

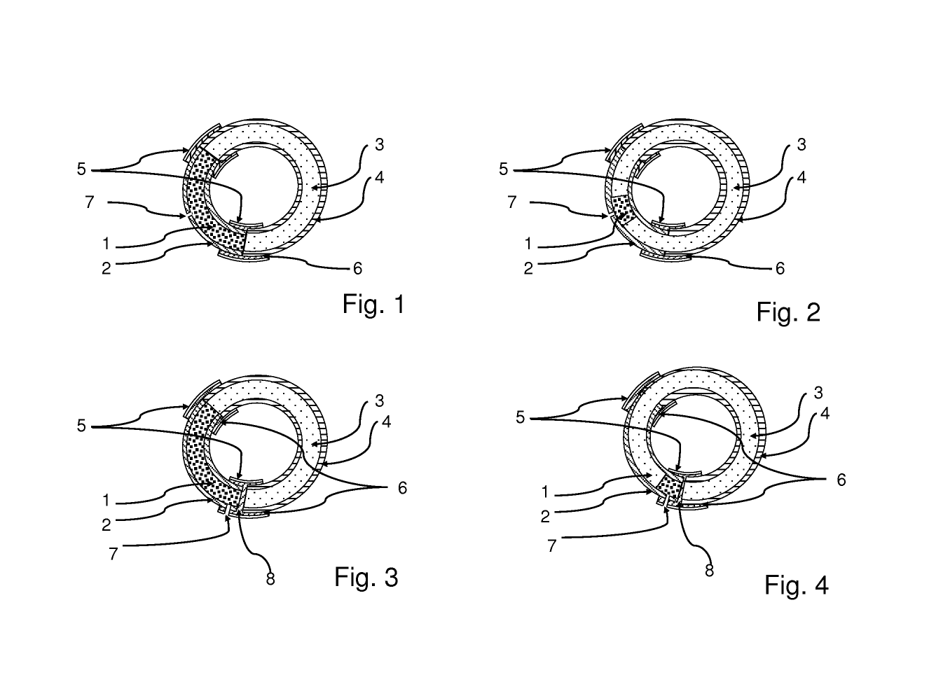

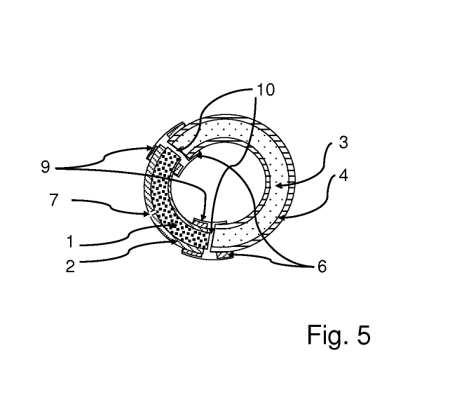

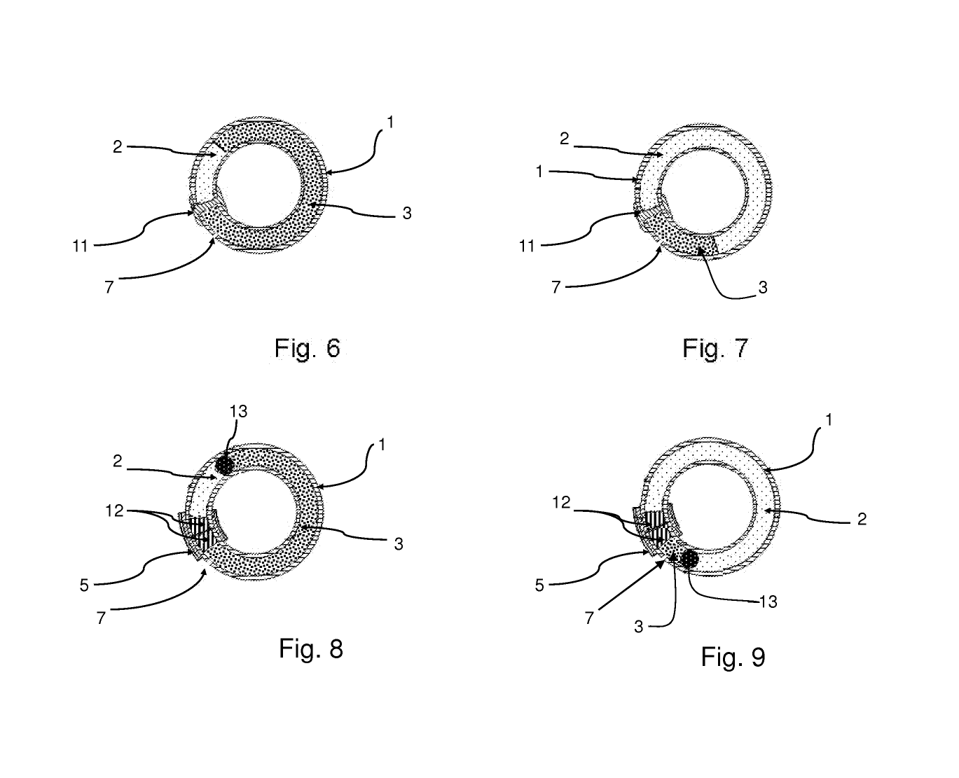

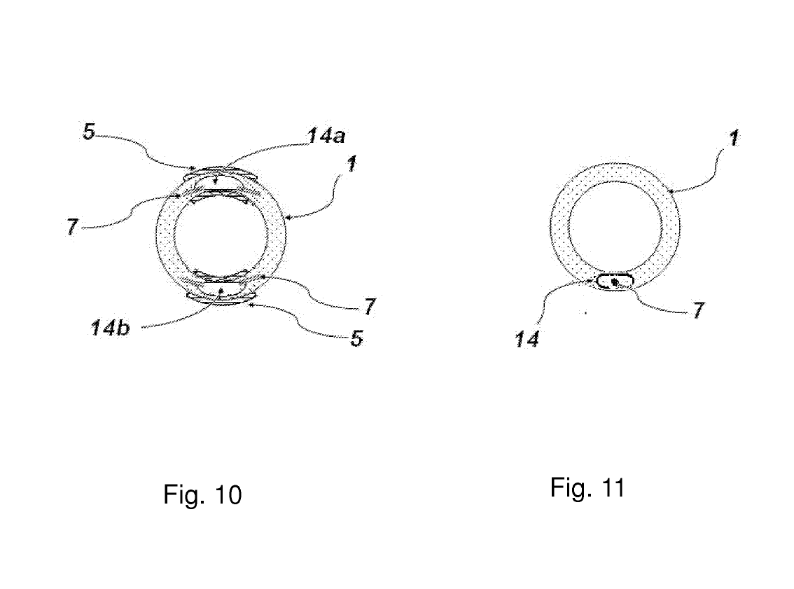

### DETAILED DESCRIPTION OF THE INVENTION

The present invention provides an osmotically active vaginal delivery system, the body of which comprises
at least one compartment comprising a composition of one or more therapeutically active substances
at least one compartment, either the same or different from the one comprising the therapeutically active substance(s), which comprises an osmotical composition capable to interact with water and/or aqueous biological fluids to create a concentration gradient against the exterior fluid or to swell or expand to create osmotic pressure
at least one passageway which extends from the compartment(s) comprising the composition of one or more therapeutically active substance(s) to the outer surface of the body, and
optionally one or more membranes covering at least part of the delivery system, wherein the membrane is permeable to the passage of water or external aqueous fluid present in the vaginal cavity but is impermeable to the compositions inside the system.

According to an embodiment of the invention, the vaginal delivery system comprises a body and one compartment, said compartment comprising an osmotic composition and a composition of one or more therapeutically active substances. The delivery system further comprises at least one passageway extending from the compartment to the outer surface of the delivery system.

According to another embodiment of the invention the vaginal delivery system comprises a body and two compartments, one compartment comprising a composition of one or more therapeutically active substances and the other compartment comprising an osmotical composition capable to interact with water and/or aqueous biological fluids to create a concentration gradient against the exterior fluid, or to swell or expand to create osmotic pressure, and at least one passageway extending from the compartment comprising the composition of one or more therapeutically active substance(s) to the outer surface of the delivery system.

According to a further embodiment of the invention, the vaginal delivery system comprises a body and at least one compartment comprising a composition of one or more therapeutically active substances, at least one compartment, either the same or different from the one comprising the therapeutically active substance(s), which comprises an osmotic composition, at least one passageway extending from the compartment comprising the composition of one or more therapeutically active substance(s) to the outer surface of the delivery system, and one or more membrane layers covering at least part of the delivery system.

The body of the delivery system comprises a polymer composition which is permeable to the passage of water or external aqueous fluid present in the vaginal cavity but is impermeable to the compositions inside the system. The polymer composition of the body is either a polymer matrix with the compartment or compartments located therein in the form of cavities having a preselected size, or a tubular polymer wall which defines the outer wall of the compartment or compartments. The tubular body may be at least partly be filled by a polymer composition to adjust the mechanical properties of the device and/or the size of the compartments.

Optionally the delivery system comprises at least one membrane layer made of a suitable polymer composition which is permeable to the passage of water or external aqueous fluid present in the vaginal cavity but is impermeable to the compositions inside the system (i.e. said membrane is semipermeable). The membrane may cover the whole delivery system or cover only a part of the system, whereby the degree of extension can vary. In a further embodiment the membrane layer(s) are tubular polymer segments having essentially equal or slightly greater inner diameter than the outer diameter of the compartments. When manufacturing the delivery system, the ends of the compartment(s) are for example inserted into these segments to form the ring shaped vaginal delivery system.

When the membrane layer covers a part of the delivery system, the compartment, especially the compartment comprising a composition of one or more therapeutically active substances and an osmotic composition, for example an osmotic capsule like GITS (gastrointestinal therapeutical system), can be introduced inside this membrane.

A preferred embodiment according to the invention is a vaginal delivery system wherein the composition containing the therapeutically active substance is pressed to a solid form having a preselected shape (tablet) and is located in the same compartment as the osmotic composition. The osmotic composition is preferably mixed with the active substance before pressing to a solid form, and the obtained osmotic tablet is covered with a semipermeable membrane comprising an outlet passageway. The passageway of the body of the intravaginal delivery system is placed to match the passgeway of the membrane.

In another preferred embodiment wherein the therapeutically active substance is pressed to a solid form having a preselected shape and is located in the same compartment as the osmotic composition, the osmotic composition is either in the form of a layer surrounding the active substance or is placed inside the active substance, and the solid combination of the two compositions is surrounded by a semipermeable membrane comprising an outlet passageway. The passageway of the body of the intravaginal delivery system is placed to match the passageway of the membrane.

In another preferred embodiment wherein the therapeutically active substance and the osmotic composition are in the same compartment, a layer comprising the osmotic composition and a layer comprising the therapeutically active substance are bonded together by compression to form a tablet-shaped core which is coated by a semipermeable membrane. The semipermeable membrane comprises an outlet passageway on the drug layer side of the tablet. The passageway of the body of the intravaginal delivery system is placed to match the passageway of the membrane.

The polymer composition of the body, membrane or the material used to fill the body consists of a material which is permeable to the passage of water or an external aqueous fluid present in the vaginal cavity so as to retain water flux rate in the desired range, but is substantially impermeable to passage of the compositions inside the system so that osmogents or therapeutically active substances or ions are not lost by diffusion across the delivery system and the undesired movement of active substance from parts of the body containing active substance to parts free of active substance during storage take place only very slowly.

The polymer composition should be stable both to the outer and the inner environment of the device. It must be sufficiently rigid to retain its dimensional integrity during the operational lifetime of the device, and finally, it must be biocompatible.

The materials of the polymer composition are preferably pharmaceutically acceptable elastomers selected from the group of siloxane polymers, polyurethane (PU, PUR), ethylene-vinyl acetate copolymer (EVA), hydrocarbon polymers such as polyisobutylene, styrene-butadiene-styrene block copolymeres (SBS, SIS), styrene-isoprene-butadiene-styrene copolymers (SIBS) and other polyolefins. For the swellable compartments, PU or siloxane polymers are preferably used because of their high water vapour transmission rate (WVTR). This permits rapid uptake of water vapour into the swellable matrix at the site of vaginal application.

However, it is also possible to use thermosetting plastics such as polyester or polycarbonate, unplasticized cellulose acetate, plasticized cellulose acetate, reinforced cellulose acetate, cellulose di- and triacetate, ethyl cellulose and the like.

The osmotic composition is preferably distant from the passageway. The compartments or compositions may be in contact with each other, but they may as well be separated by a biocompatible membrane or barrier layer impermeable to the compositions of the system to prevent the compositions from coming into contact with each other. The impermeable membrane or barrier layer may be for example in the form of a polymer layer, air gap, or a ball or a cylinder made of steel, titanium, glass or Teflon. Suitable barrier polymers are known to a person skilled in the art, e.g. Barex or Surlyn, which are used for packaging in the food industry or in pharmaceutical products, or steel, titanium, glass, Teflon or like. The ends of an originally rod formed polymer composition can during manufacturing be firmly connected to each other by adapter pieces, which prevent direct contact of the compositions in the interior of the delivery system. The adapter pieces are preferably made of a biocompatible material that constitutes a diffusion barrier for pharmaceutical active substances, for example chosen from the group of Teflon, siloxane polymers, copolymers of Teflon and siloxane polymers, polyacrylonitrile and olefins.

The compositions can be in the form of a gel, paste or suspension or in liquid, semisolid or solid state and may, in addition to the therapeutically active or osmotically active substances, comprise pharmaceutically acceptable excipients and/or carriers. When the composition comprising the active substance is present in a solid or semi-solid, non-free flowing state at a temperature of 25° C., it preferably adopts a liquid form at a body temperature of 37° C.

The therapeutically active composition may be soluble in the exterior fluid and itself exhibit an osmotic pressure gradient across the body material against the fluid. Completely insoluble or only sparingly soluble active substances are generally admixed or used together with an osmotic composition capable of generating the required osmotic pressure against the fluid.

When the delivery system is placed in the vagina, water or exterior aqueous fluid is absorbed through the body material or the tubular polymer segment. The absorption of water into the osmotic composition may also occur by water vapour transmission through said materials. As a result, the osmotic composition expands thereby forming a formulation, a solution or suspension comprising the therapeutically active composition that will be released through the at least one passageway at a constant rate. The release is driven by the concentration gradient against the exterior fluid. When the device comprises separate compartments for the composition containing an active agent and the osmotic composition, the latter functions as an expandable driving member and operates to diminish the volume occupied by the active agent, thereby delivering the agent from the device at a controlled rate over an extended period of time. The active substance will be released from the device in the form of a solution and/or suspension.

The release rate can generally be adjusted through water permeability of the polymer composition, the area through which water is absorbed, thickness of the material, size and number of passageways, and selection of the osmotic composition. Since the selected polymer composition is substantially impermeable to passage of the compositions from inside the system, the release of the active substance does not or only to a negligible extent take place through diffusion and is therefore not dependent on the diffusion coefficient of an active substance in the polymer composition.

Within the dimensions of a typical vaginal ring the compartment or compartments can have any length or size, which is not intended to be limited by the figures shown. The size of the compartment(s) and the load of each composition in the compartments will be chosen based on the intended use of the delivery system. In general, a higher load of the therapeutically active substance permits a longer period of delivery or higher dosage of the substance released from the system, whereas a higher load of the osmotic composition leads to increased concentration gradient, swelling or expanding in the respective part of the ring, as a result of which the composition comprising the active substance will be forced more quickly out of the delivery system. For example, when the delivery system is intended to release the active substance within a period of from couple of hours to 7 days, the amount of osmotic agent should be higher than the amount of therapeutically active substance in a system having both compositions in the same compartment, or the compartment comprising the osmotic composition should be larger than the compartment comprising the therapeutically active substance when the compositions are in separate compartments. Respectively, if the active substance is to be released over a longer time, from one week to several months, the amount of the active substance should be higher than the amount of osmotic agent in a system having both compositions in the same compartment, or the compartment comprising the active substance should be larger than compartment comprising the osmotic composition when the compositions are in separate compartments.

The delivery system comprises at least one passageway extending from the inside of the compartment comprising the composition with the active substance to the outer surface of the body of the delivery system to permit effective release of the therapeutically active substance to the exterior of the system. Thus the composition with active substance is close to the passageway, and the osmotic composition is positioned distant from the passageway.

The term passageway, as used herein comprises means and methods suitable for releasing the agent or drug from the osmotic system and includes one or more aperture, orifice, hole, porous element, hollow fiber, microchannel, capillary tube, microporous insert, pore, microporous overlay, or bore, and the like, through the body or the membrane of the device to the compartment(s) comprising the therapeutically active substance. The passageway can be formed e.g. by mechanical drilling, laser drilling, eroding an erodible element, extracting, dissolving, by an indentation or by using leachable substances in the permeable wall or by other appropriate techniques known in the art. Laser drill is well established for producing sub-millimeter size holes. The passageway can have any shape such as round, triangular, square, elliptical, and the like. When the active substance containing compartment is in a solid form having a permeable coating or encasing membrane comprising a separate outlet passageway, the passageway of the body is preferably placed above the solid composition and passageways placed to match each other.

A wide variety of compositions known to a person skilled in the art can be used as the osmotic composition, i.e. compositions capable to interact with water and aqueous biological fluids to create the concentration gradient against the exterior fluid or to swell or expand to create osmotic pressure.

The osmotically effective compounds or osmotically effective solutes can be used by mixing them with a therapeutically active agent, or with an osmopolymer to form a composition containing the therapeutically active agent that is osmotically delivered from the device.

The osmotically effective polymers can also be used as such in a delivery system comprising a separate compartment for the therapeutically active substance to create a hydrostatic pressure needed to drive the fluid or suspension of said substance out through the passageways to the target organ. The osmotic solutes are used by homogeneously or heterogeneously mixing the solute with the agent or osmopolymer and then charging them into the reservoir. The solutes and osmopolymers absorb fluid into the reservoir producing a solution of solute in a gel which when delivered from the system transport undissolved or dissolved therapeutically active substances to the exterior of the system.

Water-soluble compounds suitable for inducing osmosis, i.e. osmotic agents or osmogents, include all pharmaceutically acceptable and pharmacologically inert water-soluble compounds referred to in the pharmacopeias. The examples of agents used for inducing osmosis include inorganic salts such as magnesium chloride or magnesium sulphate, lithium, sodium or potassium chloride, lithium, sodium or potassium hydrogen phosphate, lithium, sodium or potassium dihydrogen phosphate, potassium sulfate, sodium sulphate, sodium sulphite, sodium carbonate, lithium sulphate, salts of organic acids such as sodium or potassium acetate, magnesium succinate, sodium benzoate, sodium citrate or sodium ascorbate; magnesium succinate, tartaric acid, carbohydrates such as mannitol, sorbitol, xylitol, arabinose, ribose, xylose, glucose, fructose, mannose, galactose, sucrose, maltose, lactose, raffinose; alpha-d-lactose monohydrate, water soluble amino acids such as glycine, leucine, alanine, or methionine, urea and the like, and mixtures thereof.

The osmopolymers suitable for forming the osmotic composition are hydrophilic polymers which interact with water and aqueous biological fluids and swell or expand to an equilibrium state. The polymers exhibit the ability to swell in water and retain a significant portion of the imbibed water within the polymer structure. The polymers swell or expand to a very high degree, usually exhibiting a 2 to 50 fold volume increase. The polymers can be noncross-linked or cross-linked and can be of plant, animal or synthetic origin. Examples of organic polymer osmogents include for example cellulose polymers such as sodium carboxymethyl cellulose, hydroxypropylmethyl cellulose, polyethylene oxide, vinyl pyrrolidone polymers such as crosslinked polyvinylpyrrolidone or crospovidone, copolymers of vinyl pyrrolidone and vinyl acetate, poly(hydroxy alkyl methacrylate), anionic and cationic hydrogels; polyelectrolyte complexes; poly(vinyl alcohol), a water insoluble, water swellable copolymer produced by forming a dispersion of finely divided copolymer of maleic anhydride with styrene, ethylene, propylene, butylene or isobutylene, water swellable polymers of N-vinyl lactams, and the like. Other osmopolymers include polymers that form hydrogels such as acidic carboxy polymers, polyacrylamides, polyacrylic acid, polyethylene oxide polymers and higher; starch graft copolymers, acrylate, polysaccharides composed of condensed glucose units such as diester cross-linked polyglucan, agar, alginates, carrageenan, guar gum, microbial polysaccharides such as dextran, gellan gum, xanthan gum, and the like. The polymeric swelling agent may comprise one or more of the above swellable hydrophilic polymers. Often, a mixture of two hydrophilic polymers provides the desired controlled swelling. The osmagent is usually present in an excess amount, and it can be in any physical form, such as particle, powder, granule, and the like. Particular preference is given to mixtures of high-molecular-weight polyethylene oxide (PEO), hydroxypropylmethylcellulose (HPMC) and saline solution (NaCl).

The delivery system can be used for a large number of active substances from very different classes of therapeutically active substances, including highly hydrophilic and highly lipophilic substances. The active substances can be soluble to water or aqueous fluid, but they can also be sparingly soluble or insoluble.

The term therapeutically active substance, as used herein includes any beneficial agent or compound, or prodrug thereof, that can be delivered from the delivery system into the vaginal cavity to produce a desired prophylactic or therapeutic result. The agents can be organic or inorganic, hydrophilic or lipophilic as long as they are suitable for vaginal administration and exert their effect either locally or systemically. The solubility of the substance in the exterior fluid can vary from insoluble to very soluble. Typical drugs include, without limitation, proteins such as peptides and polypeptides, RNA- or DNA-based molecules, vaccines, and combinations thereof.

The compositions chosen for at least the compartment containing therapeutically active substance are preferably those that adopt a free-flowing state under the influence of moisture uptake or under elevated temperature at the site of vaginal application. The therapeutically active substance can be in various forms in the composition, such as uncharged molecules, molecular complexes, pharmacologically acceptable salts known in the art, esters, ethers and amides. For acidic substances, salts of metals, amines or organic cations; for example, quaternary ammonium can be used. Water insoluble substances can be used in a form of a water soluble derivative, which on its release from the system is converted to the original biologically active form for example by enzymatic cleavage, hydrolysis, change of pH or other metabolic processes. The therapeutically active substance can be in dissolved or undissolved form or in suspended form. The at least partially undissolved, suspended form is preferred, since larger amounts of active substance can in this way be introduced in the system.

The composition may further comprise additional pharmaceutical excipients including, but not limited to, excipients used in producing solid formulations and granules, e.g. binders, lubricants, glidants, dispersants, colorants, diluents or fillers, compression excipients, glidants and the like, as well as material suitable to be used as coatings.

The amount of drug incorporated in the osmotic device varies widely depending on the particular drug, the desired therapeutic effect, and the time span for which it takes the drug to be released. Since the dimensions and relative proportions of the compartments as well as the drug load can be changed to provide dosage regimes for various therapies, there is no critical upper limit on the amount of drug incorporated in the device. Also the lower limit will depend on the activity of the drug and the same time span of its release. Thus it is not practical to define a range for the therapeutically effective amount of drug to be released by the device.

The delivery system may be provided with a means to check the point when the therapeutically active substance has completely been delivered. The means may for example include different and easily distinguishable colours of the composition comprising the active substance and the osmotic composition. In a preferred embodiment, the active agent and the hydrophilic polymer have contrasting colors. The body may be made sufficiently transparent to permit easy observation of the colour.

Osmotically active delivery systems can be manufactured by methods known in the art. For example, polymer composition can be extruded to form a core or a tube, which is filled with the compositions of therapeutically and osmotically active agents by a desired way to form the body of the delivery system. Finally the end pieces of the body comprising the compartment(s) are connected to form a delivery system suitable for vaginal administration, preferably a vaginal ring, for example by inserting the end-pieces of the body into tubular polymer segment(s), i.e. a polymer tube or polymer tubes having a suitable length and an inner diameter which is essentially equal or slightly larger than the outer diameter of the body and then by completely sealing the ends by a composite adhesive. The ends of a tube-formed body can also be connected by using suitable adapter piece(s) having a diameter corresponding the internal diameter of the tubular body. The adapter pieces may consist of a material which prevents direct contact of the compositions in the interior of the delivery system.

The passageway is made by using for example a needle or laser drilling.

The active substance can be mixed with an osmotic composition and excipients, and pressed into a solid having dimensions that correspond to the internal dimensions of the body. The active substance and other formulation forming ingredients and a suitable solvent can also be mixed into a solid or a semisolid by conventional methods such as ballmilling, calendering, stirring or rollmilling, and then pressed into a preselected shape. Next, a layer of a composition comprising an osmotic composition is laced in contact with the layer of active substance formulation, and the two layers are surrounded with a polymer composition. The layering can be accomplished by conventional two-layer tablet press techniques. The wall can be applied by molding, spraying, or dipping the pressed shapes into wall-forming materials.

A solid composition can be inserted in the membrane tube or in a tubular polymer segment, whereafter the ends of the tube or the body, respectively, are connected as described above. To adjust or modify the mechanical properties of the device, the membrane tube can be at least partly filled with a suitable polymer composition.

### EXAMPLE 1

### Manufacture of an Osmotically Active Polyurethane Capsule

The resin and the catalyst of a two component resin are mixed together in the ratio 1:1 (bredderpox® R12GB von Breddermann). When the exothermic reaction begins, a magnetic stirring rod with a diameter of 8 mm is dipped 3-4 centimeters in the mixture for 2-3 hours to get a thin layer of resin on the rod. After the resin has thoroughly hardened (approximately 12 hours) the capsule will be cut to the length of 3 cm. The counterpart for the capsule is made in a similar way by using a rod with a diameter of 6 mm. This capsule will be filled with 250 mg of the osmotic composition comprising ferric oxide as a colorant (0.977 wt-%), hydroxypropyl methylcellulose (5.006%), magnesium stearate (0.244%), polyethylene oxide (64.591%) and sodium chloride (29.182%). The larger capsule is used as a cap, sealed with an adhesive and finally the remaining space in the capsule is filled with the composition containing 19.22 mg of the active substance ZK 246965 (11β-Fluoro-17α-methyl-7α-{5-[methyl(8,8,9,9,9-pentafluorononyl)amino]pentyl}estra-1,3,5(10)-triene-3,17β-diol) in the mixture of Labrafil and Labrasol (in ratio 7:18) by injecting it through a small orifice drilled in the capsule.

### EXAMPLE 2

### Manufacture of Osmotically Active Siloxane Capsules

Elastosil A and B (Elastosil M 4641 A and 4641 B) in the ratio of 10:1 are mixed with 10 parts of cyclohexane. A glass rod with a diameter of 8 mm is dipped in the mixture to get a thin layer of polymer on the rod, and after complete polymerization the capsule will be cut to the length of 3 cm. The capsule will be filled with 500 mg of the osmotic composition, closed by a siloxane plug having the same diameter and having an orifice in the middle of this plug, sealed with an adhesive. Finally the remaining space in the capsule is filled with the composition containing 28.08 mg of the active substance ZK 246965 (11β-Fluoro-17α-methyl-7α-{5-[methyl(8,8,9,9,9-pentafluorononyl)amino]pentyl}estra-1,3,5(10)-triene-3,17β-diol) in the mixture of Labrafil and Labrasol by injecting it through the orifice.

### EXAMPLE 3

### Manufacture of an Osmotically Active Vaginal Ring

A ring formed system is manufactured, one using a polyurethane tube (Noreflex PUR 401 MHF from Norres) having inner diameter of 2 mm and outer diameter of 4 mm. Tube of 12 centimeters is processed in a ring form by sealing the ends of the tube by a two component adhesive (bredderpox® R12GB von Breddermann). By avoiding the formation of air bubbles each tube is filled with 100 mg of the osmotic composition comprising ferric oxide as a colorant (0.977 wt-%), hydroxypropyl methylcellulose (5.006%), magnesium stearate (0.244%), polyethylene oxide (64.591%) and sodium chloride (29.182%,) and with 404 mg of the composition containing 8.08 mg of the active substance ZK 246965 (11β-Fluoro-17α-methyl-7α-{5-[methyl(8,8,9,9,9-pentafluorononyl)amino]pentyl}estra-1,3,5(10)-triene-3,17β-diol) by injecting the compositions through a small orifice drilled in the ring.

### EXAMPLE 4

### Manufacture of an Osmotically Active Vaginal Ring

A ring formed system is manufactured by using a siloxane tube (60 Shore Art.-Nr. 707112020050 from ESSKA GmbH) having inner diameter of 2 mm and outer diameter of 4 mm. A tube of 12 centimeters is processed in a ring form by sealing the ends of tubes by a two component adhesive (Elastosil M 4641 A and Elastosil M 4641 B). By avoiding the formation of air bubbles each tube is filled with 100 mg of the osmotic composition comprising ferric oxide as a colorant (0.977 wt-%), hydroxypropyl methylcellulose (5.006%), magnesium stearate (0.244%), polyethylene oxide (64.591%) and sodium chloride (29.182%,) and with 473 mg of a composition containing 9.46 mg of the active substance ZK246965 (11β-Fluoro-17α-methyl-7α-{5-[methyl(8,8,9,9,9-pentafluorononyl)amino]pentyl}estra-1,3,5(10)-triene-3,17β-diol) by injecting the compositions through a small orifice drilled in the ring.

### EXAMPLE 5

The osmotically-controlled vaginal delivery system was prepared according to the general description given below.

Silica-filled silicone elastomer was formed to a sheet and crosslinked in a laboratory hydraulic press at 200° C. using a pressure of 100-200 bar. After that the elastomer was further cured for 1.5 h in a vacuum oven at 105° C. using a reduced pressure of ca 200 mbar. The sheets were pressed to thicknesses of 0.5, 1.0, and 2.0 mm. The formed sheets were cut into round pieces using a punch and, if applicable, a round hole was punched in the pieces.

The tablets containing the beneficial agent were pushed in the pre-made hole of the silicone elastomer sheet in the way that the elastomer formed a frame around the side of the tablet. A top and bottom sheet was glued to the elastomer frame, using silicone adhesive, in order to completely embed the tablet in the silicone elastomer. An embedded tablet is shown in the figure below.

TABLE 1
Tablets embedded in silicone elastomer

| Bottom-membrane | Top-membrane | Tablet |
|---|---|---|
| 0.5 mm thick | 0.5 mm thick; | GITS 20 mg |
| | 2 mm hole | coated |
| 0.5 mm thick; | 0.5 mm thick; | GITS 20 mg |
| 4 mm hole | 2 mm hole | coated |
| 1.0 mm thick | 1.0 mm thick; | GITS 20 mg |
| | 2 mm hole | coated |
| 0.5 mm thick | 0.5 mm thick; | GITS 30 mg |
| | 2 mm hole | coated |
| 0.5 mm thick | 0.5 mm thick; | GITS 60 mg |
| | 2 mm hole | coated |
| 0.5 mm thick | 1.0 mm thick; | GITS 60 mg |
| | 2 mm hole | coated |
| 1.0 mm thick | 1.0 mm thick; | GITS 30 mg |
| | 2 mm hole | coated |
| 1.0 mm thick | 1.0 mm thick; | GITS 60 mg |
| | 2 mm hole | coated |
| 0.5 mm thick; | 0.5 mm thick; | GITS 60 mg |
| 4 mm hole | 2 mm hole | coated |
| 0.5 mm thick | 1.0 mm thick; | GITS 20 mg |
| | 0.5 mm hole | uncoated |
| 0.5 mm thick | 2.0 mm thick; | GITS 20 mg |
| | 0.5 mm hole | uncoated |

### EXAMPLE 6

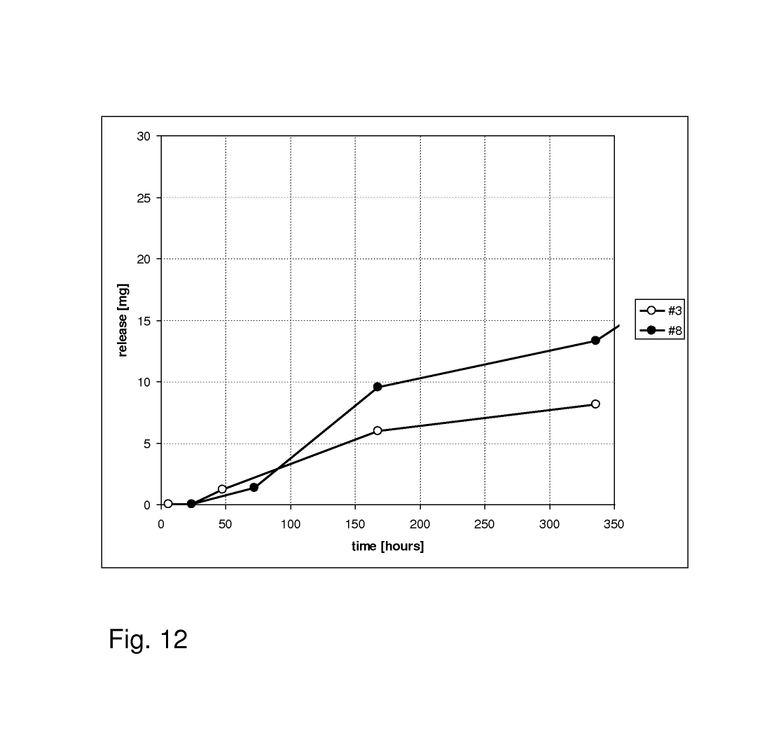

The osmotically-controlled vaginal delivery systems #3 and #8 prepared according to Example 5 were subjected to a release test. The initial concentration of the beneficial agent, 20 mg (#3) or 60 mg (#8), does not have a significant influence on the release rate, as can be seen in FIG. 12. A higher initial concentration of the beneficial agent will, however, offer a prolonged release profile.

### EXAMPLE 7

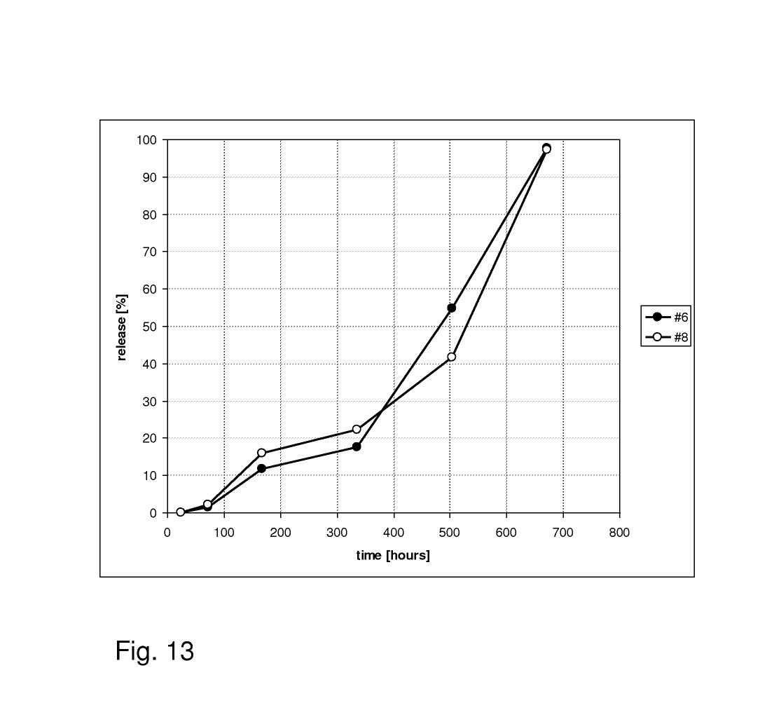

The osmotically-controlled vaginal delivery systems #6 and #8 prepared according to Example 5 were subjected to a release test. The release, converted to per cent of total concentration, is presented in FIG. 13. The experiment shows that a similar release profile is obtained regardless of the bottom membrane thickness. A thicker elastomer membrane can thus be used where a more rigid product is needed without compromising the release rate.

### EXAMPLE 8

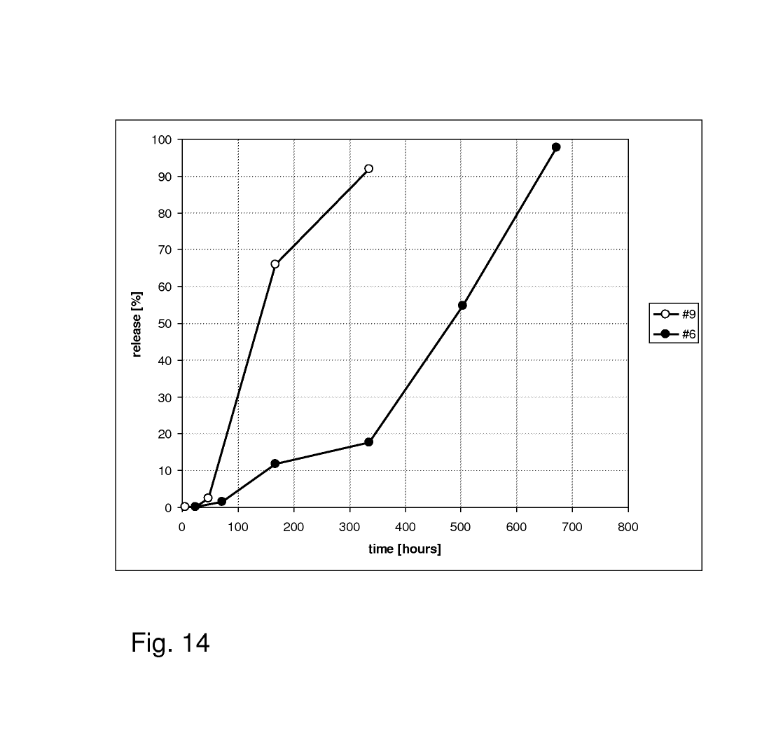

The osmotically-controlled vaginal delivery systems #6 and #9 prepared according to Example 5 were subjected to a release test. The release, converted to per cent of total concentration, is presented in FIG. 14. The embedded tablet #9 is otherwise the same as the embedded tablet #6, but has a 4 mm hole in the bottom membrane for faster water uptake. The experiment shows, that the release profile can be adjusted to a desired level by controlling the water uptake into the embedded tablet.

### EXAMPLE 9

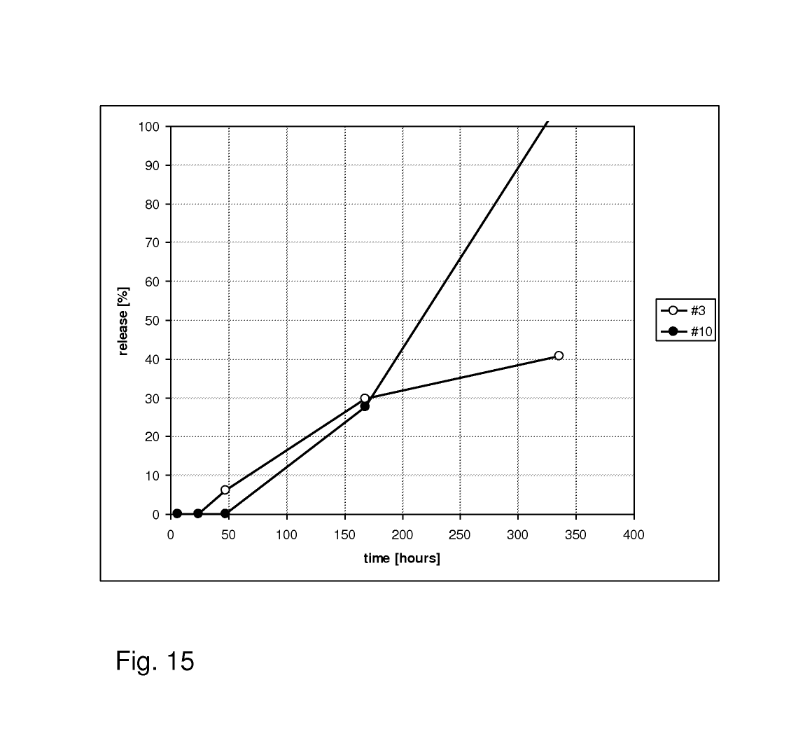

The osmotically-controlled vaginal delivery systems #3 and #10 prepared according to Example 5 were subjected to a release test. The release, converted to per cent of total concentration, is presented in FIG. 15. The experiment shows that the release profile can be greatly enhanced by using uncoated tablets.

1. An osmotically active vaginal delivery system, the body of which comprises
at least one compartment comprising a composition of one or more therapeutically active substances
at least one compartment, either the same or different from the one comprising the therapeutically active substance(s), which comprises an osmotical composition capable to interact with water and aqueous biological fluids to create a concentration gradient against the exterior fluid or to swell or expand to create osmotic pressure, and
at least one passageway extending from the compartment comprising the composition of one or more therapeutically active substances to the outer surface of the body.

2. An osmotically active vaginal delivery system according to claim 1 wherein the body of the delivery system comprises polymer composition which is permeable to the passage of water or external aqueous fluid present in the vaginal cavity but is impermeable to the compositions inside the system.

3. An osmotically active vaginal delivery system according to claim 1, wherein at least part of the delivery system is covered by a membrane made of polymer composition which is permeable to the passage of water or external aqueous fluid present in the vaginal cavity but is impermeable to the compositions inside the system.

4. An osmotically active vaginal delivery system according to claim 3, wherein said membrane is in the form of a tubular polymer segment having equal or slightly greater inner diameter than the outer diameter of the system.

5. An osmotically active vaginal delivery system according to claim 1, wherein the therapeutically active composition and the osmotical composition are in the same compartment.

6. An osmotically active vaginal delivery system according to claim 1, wherein the therapeutically active composition and the osmotical composition are in separate compartments.

7. An osmotically active vaginal delivery system according to claim 4, wherein said at least one compartment is inside the tubular polymer segment.

8. An osmotically active vaginal delivery system according to claim 6, wherein the compartments are separated by an impermeable membrane or barrier layer to prevent the composition comprising the active substance from coming into contact with the osmotic composition of the adjacent compartment.

9. An osmotically active vaginal delivery system according to claim 8, wherein said impermeable membrane or barrier layer is made of a material that constitutes a diffusion barrier for pharmaceutically active substances and is preferably chosen from the group of Teflon, siloxane polymers, copolymers of Teflon and siloxane polymers, polyacrylonitrile or olefins.

10. An osmotically active vaginal delivery system according to claim 8, wherein the impermeable membrane or barrier layer is in the form of a ball or a cylinder made of steel, titanium, glass or Teflon.

11. An osmotically active vaginal delivery system according to claim 1, wherein the composition comprising the active substance is present in a solid form in the same compartment as the osmotic composition, the compartment is surrounded by a semipermeable membrane, and the semipermeable membrane comprises a passageway placed to match a passageway of the body of the intravaginal delivery system.

12. An osmotically active vaginal delivery system according to any of the preceding claims, wherein the body material constitutes a diffusion barrier for pharmaceutical active substances and is preferably chosen from the group of siloxane polymers, polyurethane, polyurethane elastomers, polyacrylonitrile, ethylene-vinyl acetate copolymer (EVA), polyolefins such as polyisobutylene, styrene-butadiene-styrene block copolymeres (SBS, SIS) and styrene-isoprene-butadiene-styrene copolymers (SIBS), thermosetting plastics such as polyester or polycarbonate, cellulose acetates, ethyl cellulose and the like.
